(19) United States
(12) Patent Application Publication
(10) Pub. No.: US 2004/0106959 A1
(43) Pub. Date: Jun. 3, 2004

(54) Implantable Medical Device with Multiple Electrode Lead

(76) Inventors: John A. Schmidt, Leesburg, VA (US); William B. Rottenberg, Durango, CO (US); Mary Alaine Medio, Arlington, TX (US); Erlend M. Olson, Duarte, CA (US)

Correspondence Address: JOHN RICHARD MERKLING, 11171 WEST EXPOSITION DIRVE, LAKEWOOD, CO, 80226, US

(21) Appl. No.: 10/605,542

(22) Filed: Oct. 7, 2003

Related U.S. Application Data
(60) Provisional application No. 60/319,605, filed on Oct. 10, 2002.

(51) Int. Cl. A61N 1/18

(52) U.S. Cl. 607/11

(57) ABSTRACT

An implantable cardiac stimulator system or medical apparatus comprising an electrical device, a hermetically sealed case, a non-conducting plate in the case, and a plurality of electrical contacts on the plate. The system also has a lead having a plurality of electrodes, and a plurality of electrical conductors coupled to electrical contacts. The lead has a connector with a non-conducting sheet and electrical contacts protruding from the sheet, each of said contacts being connected to an electrical conductor. The contacts on the connector are arranged to contact one of said electrical contacts on the plate. Electrical contacts on sheet may comprise pins extending through the sheet, the pins comprising a bottom section and a flat section. The flat section extends above the sheet and is split by a slot, forming a first tab and a second tab, the second tab being longer than said first tab. One of the conductors of the lead is inserted into a slot of a pin. The lead connector has a compressible seal circumferentially surrounding a plurality of electrical contacts on the device and on the connector.

### BACKGROUND OF INVENTION

This invention pertains to cardiac stimulation devices such as pacemakers, cardiovertors and defibrillators and particularly to devices using multiple electrodes, and more particularly, to connectors for coupling multiple electrode leads to implantable devices.

The heart is a mechanical pump that is stimulated by electrical impulses.

The mechanical action of the heart results in the flow of blood. During a normal heartbeat, the right atrium (RA) fills with blood from the returning veins. The RA then contracts and this blood is moved into the right ventricle (RV). When the RV contracts it pumps that blood to the lungs. Blood returning from the lungs moves into the left atrium (LA), and after LA contraction, is pumped into the left ventricle (LV), which then pumps it throughout the body. Four heart valves keep the blood flowing in the proper directions.

The electrical signal that drives this mechanical contraction starts in the sinus node, a collection of specialized heart cells in the right atrium that automatically depolarize (change their voltage potential). This depolarization wave front passes across all the cells of both atria and results in atrial contraction. When the advancing wave front reaches the A-V node it is delayed so that the contracting atria have time to fill the ventricles. The depolarizing wave front then passes over the ventricles, causing them to contract and pump blood to the lungs and body. This electrical activity occurs approximately 72 times a minute in a normal individual and is called normal sinus rhythm.

The corresponding electrical signals identifying these events are usually referred to as the P, QRS (or R) and T waves or beats. More particularly, an atrial contraction is represented on an ECG by a P wave, a ventricular contraction is represented by an R wave and a ventricular repolarization is represented by a T wave. The atrium also repolarizes but this event (the U wave) is masked by activity in the ventricle and consequently it is not observable on an ECG.

Conventional pacemakers utilize a single or dual leads to apply pacing pulses. The dual (bipolar) lead typically includes a tip and a ring electrode. The lead is inserted in such a manner that the tip is imbedded into the cardiac muscle. A pacing pulse is then applied between the tip and the ring electrodes, thereby causing the cardiac muscle to contract. If a single unipolar electrode lead is used, the electric pulse is applied between the tip electrode and another electrode outside the heart, for example, the housing of the pacemaker. Bradycardia pacing therapy has usually been delivered through a pacing electrode implanted near the ventricular apex, that is, near the bottom of the heart. This location has been preferred not for physiologic reasons, but because most lead designs favor implantation at this site. A lead entering the right ventricle from the right atrium tends to extend into the lower apex of the ventricle where an active fixation apparatus, such as a helical corkscrew, may be used to secure the lead to the heart wall. Even if the distal tip of the lead is implanted at another location, it may be difficult or impossible to move the electrode to another location within the heart after initial implantation. The physician is thus limited to a single site for applying treatment. Bradycardia pacing therapy can be improved by delivering the stimulating pulse to a more efficient location than the ventricular apex. Studies have indicated that the abnormal contraction that results from apical pacing has long-term deleterious effects. Short-term studies using conventional pacing leads implanted in alternative locations have shown clinical improvements, but the long-term reliability of conventional pacing leads in these alternative locations is questionable and lead placement is difficult.

A single stimulating electrode, such as one available on a conventional lead, may not be implanted close enough to a physiologically preferred location in the patient's heart to cause improved cardiac efficiency when the pacemaker stimulates the heart. In fact, stimulating at the bottom end of the ventricle may diminish cardiac efficiency as compared to a wave propagated from the top of the ventricle. Moreover, an apparatus with a single electrode cannot control cardiac contraction, guide the propagation of a wave front, force a selected path for a stimulating wave front, or create a coordinated simultaneous or near simultaneous cardiac contraction of large sections of the myocardium. Such controlled contractions may result in more efficient cardiac contraction, thereby reducing the overall demand on the heart, allowing the body to alleviate the symptoms associated with inefficient blood flow. There is a need, therefore, for implantable devices, specifically cardiac stimulators, which can utilize a relatively large number of electrodes to stimulate and sense the heart in multiple locations.

Multiple electrode leads, however, are difficult to connect to implantable devices. The standard connector technology used in implantable medical devices stacks electrical contacts and insulators one after the other in a linear manner. Consequently, the manufacturing tolerances, and errors, accumulate. After a few electrical contacts, the position of electrical contacts cannot be assured to within acceptable manufacturing limits. Current technology for pacemaker lead connectors was developed about forty years ago and has become embodied in certain standard connectors. In general, however, such standard connectors are limited to one (“unipolar”) or two (“bipolar”) electrodes.

It has been suggested that similar linear connectors may be used in multiple electrode leads. Harris, for example, has suggested such configurations in several patents, such as U.S. Pat. No. 4,715,380 and U.S. Pat. No. 4,995,389. Such connectors rapidly reach practical limits to the number of electrodes and connections that can be accommodated.

### SUMMARY OF INVENTION

In view of the above disadvantages of the prior art, it is an objective of the present invention to provide an implantable cardiac stimulation system, such as a pacemaker, in which three or more electrodes are positioned in a chamber of the heart and a connector that can couple an implantable medical device and a multi-electrode lead. The connector can accommodate a large number of separate connections, for example, thirty-two, sixty-four or one hundred and twenty-eight connections.

It is an object of the present invention to provide an implantable cardiac stimulator system or medical apparatus comprising a cardiac stimulator having means for stimulating the heart of a patient, a hermetically sealed case, a non-conducting plate in the case, and a plurality of electrical contacts on said plate. The system also has a lead having a plurality of electrodes, a plurality of electrical conductors coupled to electrical contacts. The lead has a connector with a non-conducting sheet and electrical contacts protruding from the sheet, each of said contacts being connected to an electrical conductor. The contacts on the connector are arranged to contact one of said electrical contacts on the plate.

In another aspect of the invention, electrical contacts on the sheet may comprise a pin extending through the sheet, the pins comprising a bottom section and a flat section. The flat section extends above the sheet and is split by a slot, forming a first tab and a second tab, the second tab being longer than said first tab. One of the conductors of the lead is inserted into a slot of a pin.

In yet another aspect of the invention, the pins comprise a cylindrical bottom section extending below the sheet, the cylindrical bottom section having a rounded distal end. The cylindrical bottoms may press against buttons or rivet on the plate to form electrical contacts between the device and the lead.

Another object of the invention is a lead connector having a compressible seal circumferentially surrounding a plurality of electrical contacts on the device and on the connector.

Still another object or feature of the invention is a connector for a device and multi-electrode lead comprising a deformable material applied between electrical contacts of the lead and electrical contacts of the device, the deformable material being forced from between electrical contacts on the connector of the lead and on the device as the connector is attached to the device.

It is also an object of the invention to provide a connector for a multi-electrode lead having a lumen opening into the lead such that a stylet can be inserted through the lumen into the lead.

Other objectives and advantages of the invention will become apparent from the following description.

Briefly, the subject invention pertains to an implantable medical device such as a cardiac stimulator, a multi-electrode lead attached to the device, and a connector coupling the device to the lead. The term cardiac stimulator will be used herein to cover pacemakers as well as other cardiac devices such as cardioversion devices and defibrillators. The lead is inserted in the body into an organ to be sensed or stimulated, for example, into a cardiac chamber. Alternatively, the lead may be positioned in the veins, or it may be positioned externally of the heart or other organ to be stimulated or sensed. Since the lead has many electrodes, a multiple connector must be provided to couple the device to electrodes or sensors in the lead.

In a preferred embodiment, a lead having an elongated member is provided with the electrodes being formed on the elongated member. The electrodes comprise axially spaced electrodes disposed on the elongated member, each electrode being connected by a wire extending though said elongated member. The electrodes may be circumferential coils integral or continuous with the wires or may be rings connected to the wires by crimping or laser welding, for example. An electrode may also be provided at the distal end of the lead. The elongated member may be a tube housing the wires. The electrodes can be angularly spaced with respect to each about the elongated member. The tube may include an elongated cavity adapted to receive a removable stylet. The stylet may be more rigid then the lead and may be used for the implantation of the lead. After the lead is implanted, the stylet is removed.

The lead has a connector with a non-conducting sheet and electrical contacts protruding from the sheet, each of said contacts being connected to an electrical conductor. The contacts on the connector are arranged to contact one of said electrical contacts on the plate. Electrical contacts on sheet may comprise pins extending through the sheet, the pins comprising a bottom section and a flat section. The flat section extends above the sheet and is split by a slot, forming a first tab and a second tab, the second tab being longer than said first tab. One of the conductors of the lead is inserted into a slot of a pin. The lead connector has a compressible seal circumferentially surrounding a plurality of electrical contacts on the device and on the connector.

### BRIEF DESCRIPTION OF DRAWINGS

FIG. 1 shows a diagrammatic front view of a patient with an implantable device, specifically, a cardiac stimulation system.

FIG. 2 is a cross-sectional view of a heart with an implanted lead with a multiple conductor connector.

FIG. 3 is a plan view of a coil electrode.

FIG. 4 is a cross sectional view of a ring electrode.

FIG. 5 is a cross section of the multi-electrode lead of FIG. 3, taken along line 5-5.

FIG. 6 is a perspective view of an implantable medical apparatus, specifically, a cardiac stimulation system.

FIG. 7 is a perspective view of a connector.

FIG. 8 is a top perspective view of a sheet from the connector of FIG. 7.

FIG. 9 is a bottom perspective view of the sheet of FIG. 8.

FIG. 10 is a partial cross-sectional view of the implantable medical apparatus of FIG. 6, taken along line 10-10.

FIG. 11 is a perspective view of an implantable device, specifically a cardiac stimulator, without a connector attached.

### DETAILED DESCRIPTION

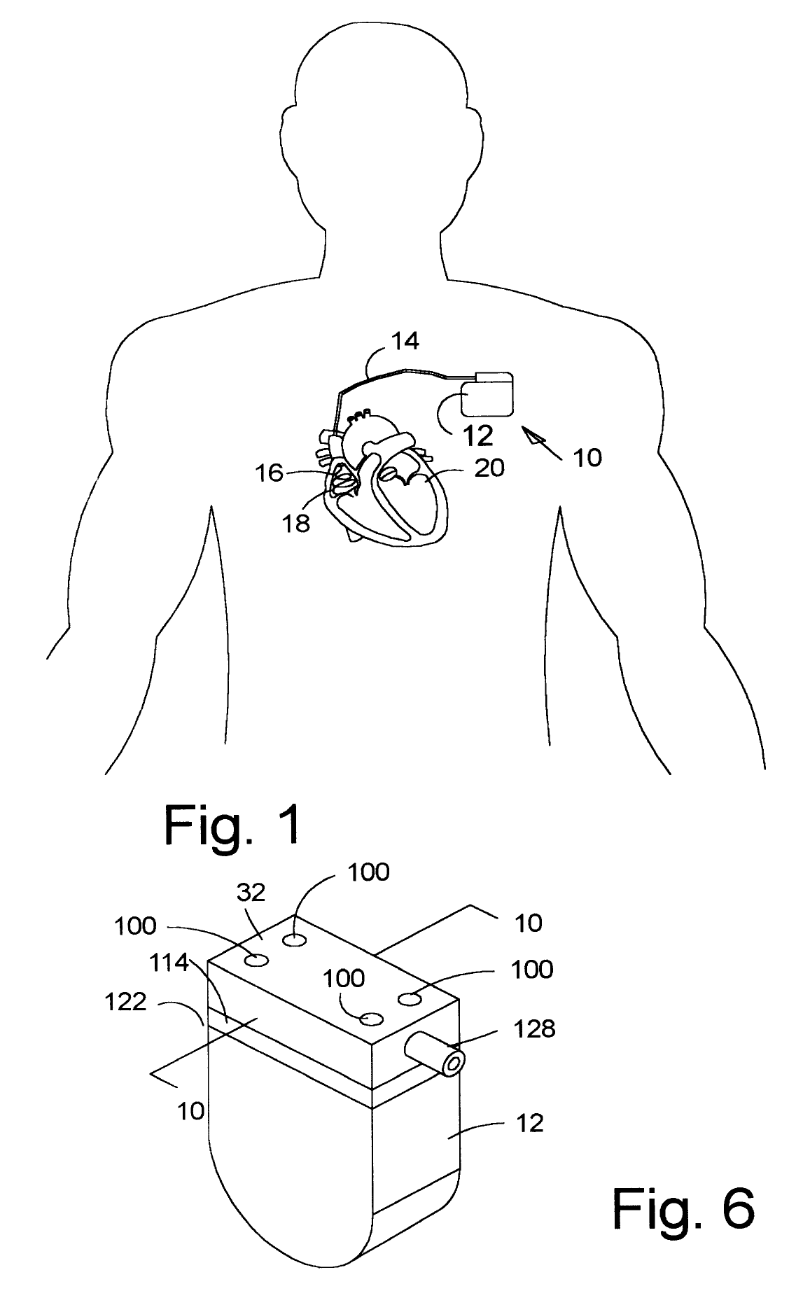

The subject invention pertains to an implantable medical device, such as a cardiac stimulation system 10 including a cardiac stimulator 12 with various electronic circuits, and a multi-electrode lead 14 attached to the stimulator 12, as shown. The lead 14 has a distal end 16 disposed, for example, in one of the cardiac chambers such as the right atrium 18 of heart 20. In FIG. 1, end 16 is shown having a general spiral shape. The system 10 is adapted to deliver therapy in the form of electrical pulses. The therapy may include GCV (greater cardiac vein) resynchronization therapy, treatment of conduction pathway abnormalities, bardycardia pacing, etc. The cardiac stimulator 12 contains electronic components common to current cardiac stimulators such as a battery, microprocessor control circuit, ROM, RAM, an oscillator, reed switch and antenna for communication, output circuits, and sense circuits. These components are well known to those of skill in the art. In addition, the cardiac stimulator 12 has a plurality of independent sensing and stimulating circuits for each heart chamber. Further details related to appropriate multi-channel sensing and stimulating circuits are found in commonly assigned U.S. patent application Ser. No. 10/134,197, filed Apr. 26, 2002, the disclosure of which is incorporated herein by reference.

Multi-Electrode Lead

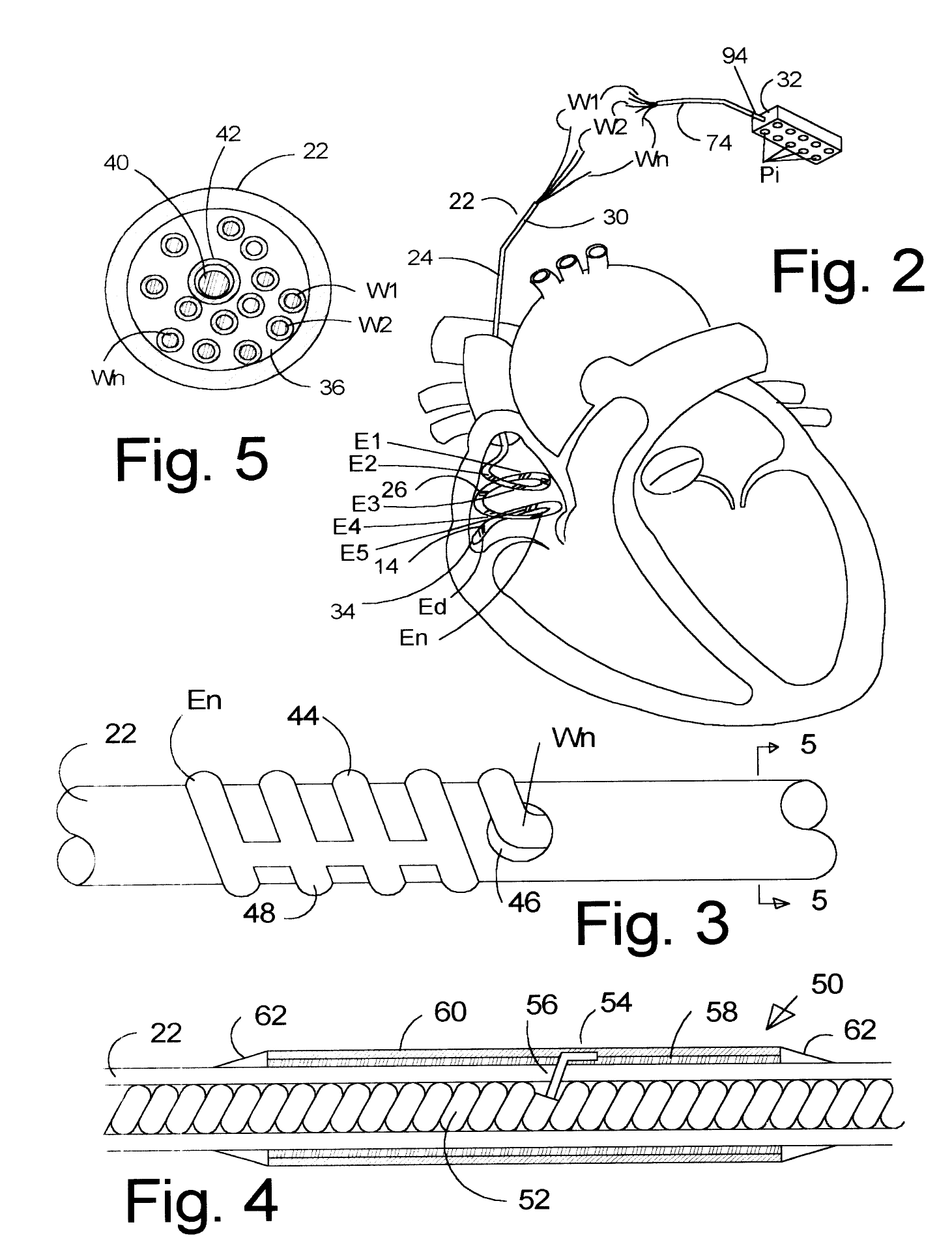

Details of the multi-electrode lead 14 are shown in FIG. 2. The lead 14 includes an external biocompatible polymer tube 22 having a straight portion 24 and a shaped portion 26. The tube may be made of polyurethane or other similar materials that may be thermally shaped so that the shaped portion 26 retains any desired configuration. In FIGS. 1 and 2, the shaped portion 26 is shown as having a spiral shape, but many other shapes may be selected as well. The spiral or coil shaped lead of FIG. 1 and 2 places electrodes around an entire chamber of the heart. This embodiment allows complete sensing and stimulating control around the entire chamber. Nevertheless, it will be apparent that numerous shapes could be selected to address the clinical needs of a particular patient.

A plurality of electrodes E1, E2, E3, E4, E5, . . . En are attached to tube 22 of the lead 14. Preferably electrodes E1. . . En are formed of coils of bare wire or cable wound about the tube 22. Each electrode is connected to corresponding wires W1, W2, W3 . . . Wn which extend through the length of tube 22 and which are shown exiting through end 30 for the sake of clarity. Wires W1, W2, W3 . . . Wn are insulated, so that they are not shorted to each other within the tube 22. The electrode 14 and its method of manufacture are disclosed in co-pending commonly assigned application Ser. No. 09/245,246 filed Feb. 5, 1999, and incorporated herein by reference. Preferably the end 30 of tube 22 and the ends of wires W1, W2, W3, etc. are coupled to a connector 32 for attaching the lead 14 to the cardiac stimulator 12. The connector 32 may have a plurality of pins Pi. Each wire W1 . . . Wn is associated with a pin. In addition to spiral coil or ring electrodes E1 . . . En, a distal tip electrode Ed may also be provided. The distal tip electrode Ed may also have an active fixation mechanism, for example a helical screw 34 or tines, to secure the lead to the interior wall of the heart.

The lead 14 can be constructed with the tube 32 extending relatively straight or can be customized to any shape to fit any pre-selected location within the heart 20 dependent on each particular patient's pathology. For example, if the lead 14 is to be placed in the greater cardiac vein, then its end 16 (consisting of tube portion 26 and electrodes E1, E2, E3 . . . etc.) is shaped to form a small helix, so that it will fit into the greater cardiac vein.

The tube 22 can be formed with a longitudinal cavity 36, as shown in the cross sectional view of FIG. 5. Cavity 36 holds the wires W1, W2, W3 etc. The lead 14 could be straightened by inserting a substantially straight stylet 40 into an interior tube or lumen 42. The stylet 40 is also flexible but is less flexible than the lead 14 so that as it is inserted into the lumen 42, it forces the tube 22 to straighten. The lead 14 is then inserted into the heart or into a vein near the heart. After implantation of the lead 14, the stylet 40 is withdrawn and the lead 14 flexes back towards the lead's original configuration.

A plurality of electrodes E1, E2, E3, E4, E5, . . . En are attached to tube 22 of the lead 14. Preferably electrodes E1 . . . En are formed of coils 44 of exposed wire or cable wound about the tube 22, as shown in FIG. 3. The wire Wn passes through a predrilled hole 46 in the tube 22. The predrilled hole 46 determines the exact location of the electrode. By changing the position and spacing of the hole, leads may be designed to cluster more electrodes along a selected segment of the lead. Since the electrodes fully circumvent the tube 22, it is likely that at least some part of the electrode will be adjacent the cardiac wall. Moreover, circumferential electrodes are unlikely to perforate the heart. Preferably the coil 44 and wire Wn are formed of one continuous wire. The loops of the coil 44 are welded 48 or otherwise connected together to provide additional structural stability. Each electrode is connected to corresponding wires W1, W2, W3 . . . Wn which extend through the length of tube 22 and which are shown exiting through end 30 for the sake of clarity. Wires W1, W2, W3 . . . Wn are insulated, so that they are not shorted to each other within the tube 22. The lead 14 is more particularly disclosed in co-pending commonly assigned application Ser. No. 09/245,246 filed Feb. 5, 1999, and incorporated herein by reference. Preferably the end 80 of tube 72 and the ends of wires W1, W2, W3, etc. are coupled to a connector 32 for attaching the lead 14 to the cardiac stimulator 12. The connector 32 may have a plurality of pins Pi. Each wire W1 . . . Wn is associated with a pin.

An alternative configuration for an electrode 50 is illustrated in FIG. 4. In this configuration, a multi-filar coil 52 comprises as many insulated-wire coils as there are electrodes on the lead. The multi-filar coil 52 lies within the tube 22. At a location of an electrode 50, an end 54 of one of the wires is passed through a hole 56 in the tube 22 and laid on an inner ring 58. A hole may also be provided in the inner ring for the wire or two inner rings may be used, one ring on either side of the wire. An outer ring 60 is placed over the inner ring or rings and crimped, capturing the end 54 of the wire between the inner and outer rings. The electrical and mechanical connection between the rings and the wire may also be improved by welding or other methods. A circumferential bead 62 of glue may seal the ends of the rings and reduce sharp edges.

In addition to spiral coil or ring electrodes E1 . . . En, a distal tip electrode Ed may also be provided. The distal tip electrode Ed may also have an active fixation mechanism, for example a helical screw or tines, to secure the lead to the interior wall of the heart.

Multi-Conductor Connector

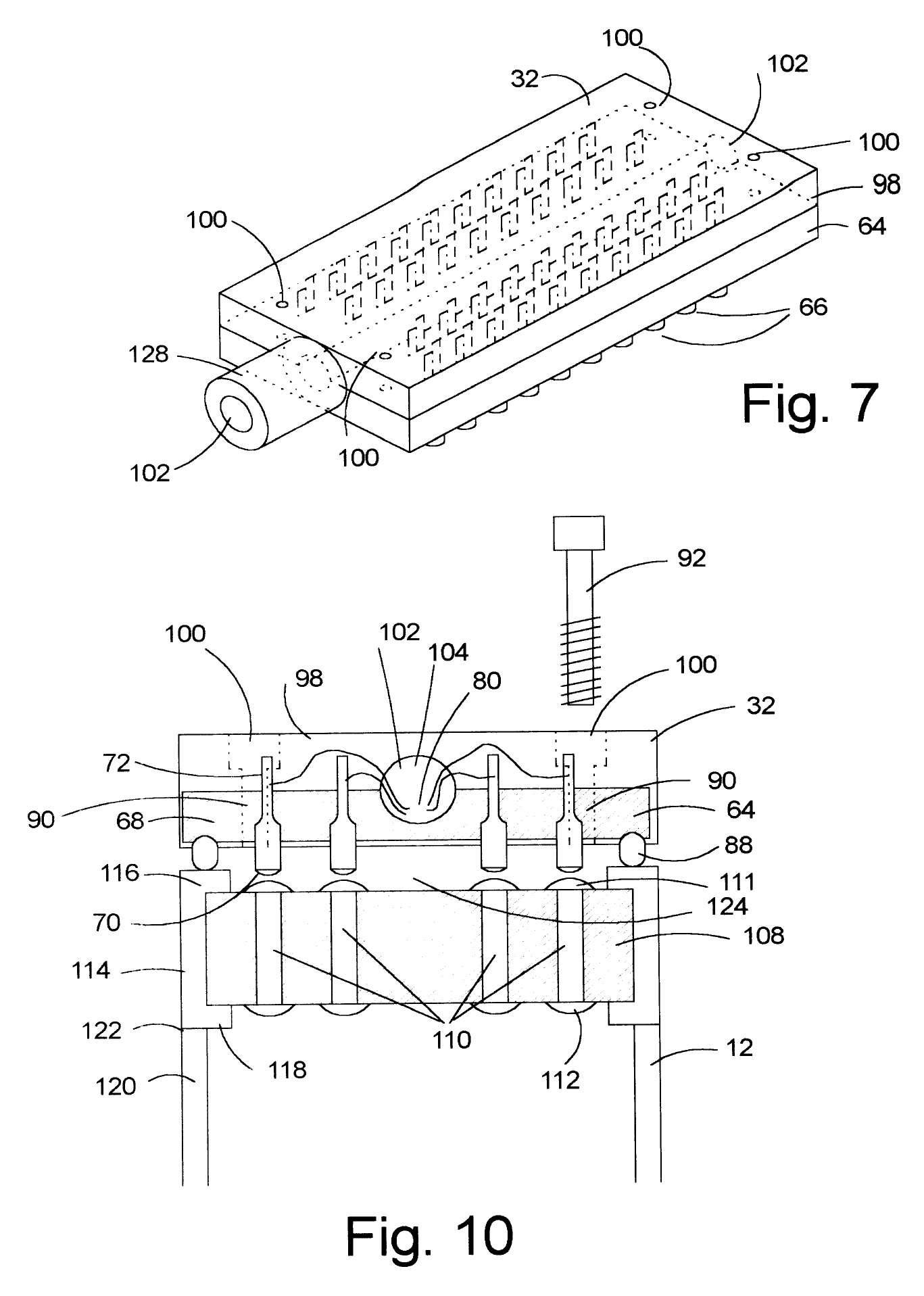

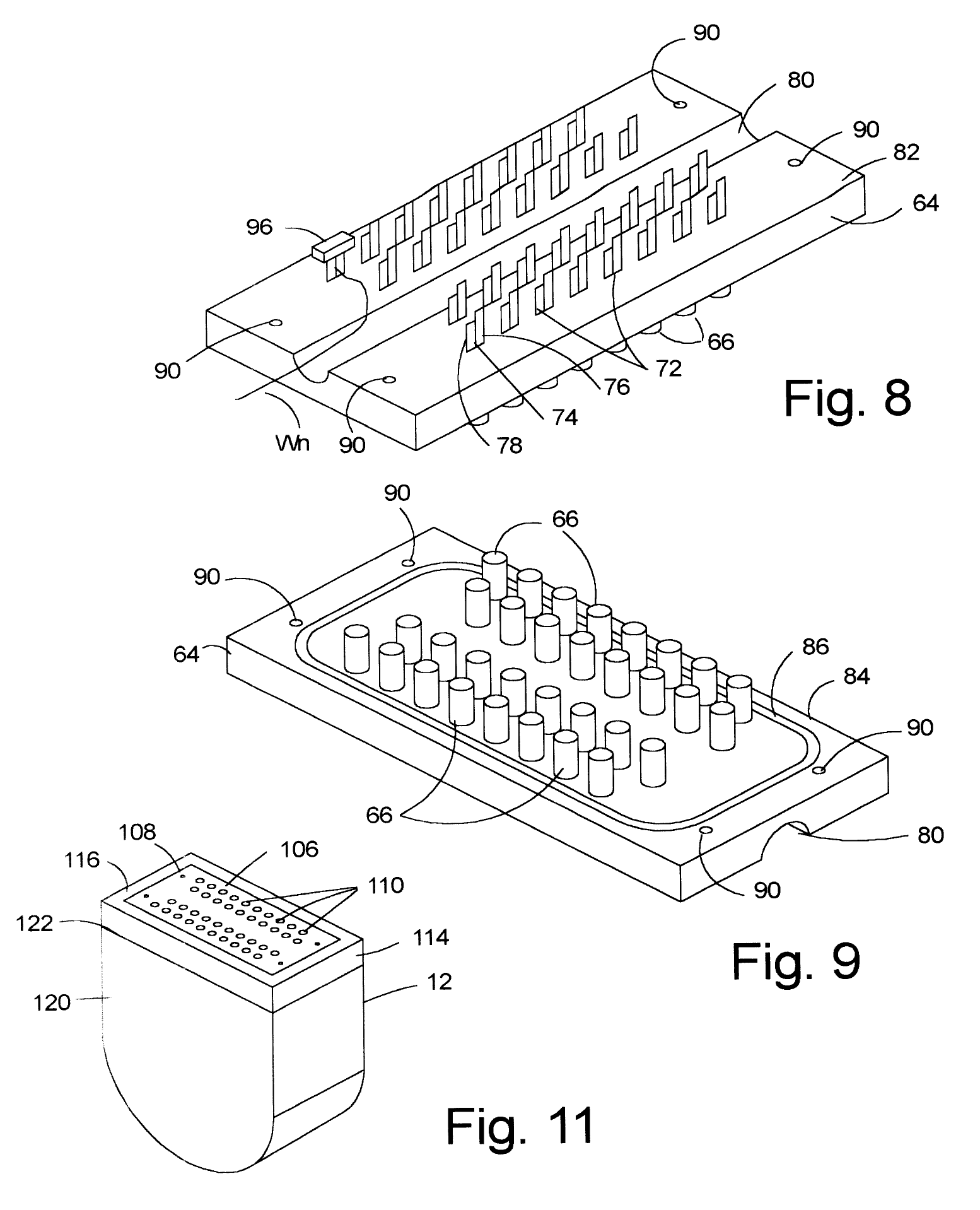

FIG. 6 shows the multi-conductor connector 32 mounted on an implantable device 12, such as a cardiac stimulator. The connector 32 comprises a non-conducting sheet 64. FIGS. 7 and 8. Mylar is an example of a suitable material. The sheet 64 supports a plurality of pins 66. The pins may be arranged in staggered rows or other configurations. In one embodiment (FIG. 10), a pin 66 comprises a cylindrical bottom section 68 with a rounded distal end 70. The bottom section 68 extends below the sheet 64. A flat section 72 extends above the sheet 64. A vertical slot 74 in the flat section 72 divides the flat section into two tabs 76, 78. A first tab 76 is taller than a second tab 78. This feature allows an assembler to place an end of a conductor against the taller first tab and slide the end of the conductor into the slot 74. Preferably, the conductor is insulated and the flat section 72 cuts the insulation as the conductor slides into the slot 74. Other methods, such as laser welding, could also be used to connect conductors to the pins.

FIG. 8 shows an arrangement of pins in the non-conducting sheet 64. By arranging four rows of eight pins, a 32 pin connector is formed. The number of pins may be selected to accommodate any desired number of conductors to support electrodes or sensors on the lead. Since the location of each pin is substantially independent of other electrodes, the pins may be placed within desired tolerances independent of the number of connections being made. This contrasts with the linear arrangement of most pacemaker or cardioverter connectors. In standard connectors, the manufacturing tolerances of each part add to each other. To accommodate a large number of connections, the individual parts must be smaller. Consequently, a limit is soon reached where it is no longer possible to assure a connection because the accumulated differences (tolerances) exceed the dimensions of a particular electrical contact.

Configurations similar to the configuration of FIG. 8 with sixty-four or one hundred twenty-eight or other numbers of connectors are possible. As shown in FIG. 8, a groove 80 may be provided in a top side 82 of the sheet 64 to accommodate a stylet to stiffen a lead during implantation. On a bottom side 84 of the sheet 64, a circumferential groove 86 receives an 0-ring or compression ring 88, shown in cross-section in FIG. 10. The corners of the groove 86 may be rounded or the groove may be ellipsoid to accept smoothly curved rings. A plurality of through bores 90 receive machine screws 92 or similar fasteners to attach the connector to the implantable device.

To assemble the connector, a proximal end 94 of the tube 22 of the lead 14 is placed in the groove 80 on the upper side 82 of the sheet. Conductors W1, W2, . . . Wn are connected individually to pins. A plastic cap 96 may be placed over the end of a flat section 72, clamping the conductor between the two tabs 76, 78. The assembly of the sheet 64, tube 22, conductors W1, W2 . . . Wn and pins 66 are inserted into a mold. Epoxy, polyurethane or similar non-conducting, biocompatible polymer encapsulates the assembly, forming a seal 98. A silicone strain relief segment 128 may be placed on the proximal end of the tube 22 and secured in the same molding process. The groove 86 for the O-ring may be molded directly in the polymer, if the bottom side 84 of the sheet 64 is also enclosed in the polymer, or the groove 86 may be provided in the sheet 64, as mentioned above.

Through bores 90 in the sheet 64 are extended through the connector as bores 100, as shown in FIGS. 7 and 10. The number and placement of bores and fasteners is a matter of ordinary design choice. As shown in the cross-sectional view of FIG. 10, the epoxy or polymer seal 98 may surround the sheet 64, conductors and pins, leaving only the rounded ends 70 of the pins 66 exposed to make electrical contact with the implantable device 12. A lumen 102 formed of the groove 80 in the sheet 64 and a corresponding hollow 104 in the epoxy seal provides access for a stylet to be temporarily inserted into the lead.

The lead connector interfaces with electrical circuitry in the implantable device 12 through a mating surface 106 shown in FIGS. 10 and 11. A non-conducting plate 108 supports a plurality of electrical contacts 110. The electrical contacts 110 are spaced to coincide with the pins 66 in the connector 32. The electrical contacts may be rivets having buttons 111, 112 on either side of the plate 108. A significant amount of pressure can be applied to the contacts 110 without failure. Pressure may be expected when the connector is secured to the implantable device 12, as explained below.

A metallic rim 114 surrounds and supports the plate 108 between an upper flange 116 and a lower flange 118. The rim 114 may be connected to a metal can 120 by a laser weld 122, forming a hermetically sealed container for batteries and electronic circuitry. Machine screws 92 attach the connector 32 to the plate 108, compressing the O-ring 88 and sealing the pins 66 and contacts 108 against body fluids. In addition, the lead connector or the mating surface 106 or both may be coated with a thin coating of non-conducting material such as parylene. As the connector is attached to the mating surface 106, pressure between the rounded ends 70 of the pins 66 and the buttons 111 forces the parylene from between the pins and buttons, allowing electrical contacts to be formed. The parylene may fill a cavity 124 formed between the connector 32 and the plate 108. Alternatively, a directionally conducting polymer foam may be used in place of parylene, such that vertical electrical conduction between contacts 108 and vertically adjacent pins 66 is permitted, but cross conduction between other contacts or pins is not.

Numerous other modifications may be made to this invention without departing from its scope as defined in the attached claims.

1. An implantable medical apparatus comprising
an electrical device having a case and a non-conducting plate in said case and a plurality of electrical contacts on said plate,
a lead having a distal end and a proximal end and carrying a plurality of electrical conductors, and
a connector coupled to a proximal end of said lead, said connector having a non-conducting sheet conformed to said plate and a plurality of electrical contacts protruding from said sheet, each of said contacts being electrically connected to an electrical conductor in said lead and being spatially arranged to contact one of said electrical contacts on said plate, and at least one fastener coupling said connector to said electrical device.

2. The implantable medical apparatus of claim 1 wherein each of said electrical contacts on said plate comprise a button exposed above said plate.

3. The implantable medical apparatus of claim 1 wherein each of said electrical contacts on said sheet comprise a pin extending through said sheet.

4. The implantable medical apparatus of claim 3 wherein said pins comprise a bottom section and a flat section, said flat section extending above the sheet and split by a slot, one of said conductors of said lead being inserted in said slot of a pin.

5. The implantable medical apparatus of claim 4 further comprising a cap on said first and second tabs, said cap clamping said conductor in said slot.

6. The implantable medical apparatus of claim 3 wherein said pins comprise a cylindrical bottom section extending below the sheet, said cylindrical bottom section having a rounded distal end.

7. The implantable medical apparatus of claim 1 further comprising a compressible seal circumferentially surrounding said electrical contacts on said plate and on said sheet.

8. The implantable medical apparatus of claim 1 further comprising a deformable material applied between said electrical contacts of said sheet and said electrical contacts of said plate, said deformable material being forced from between electrical contacts on said sheet and on said plate as said connector is attached to said electrical device.

9. The implantable medical apparatus of claim 8 wherein said material is non-conducting.

10. The implantable medical apparatus of claim 8 wherein said material is a directionally conducting polymer.

11. The implantable medical apparatus of claim 1 wherein said connector further comprises a lumen opening into said lead such that a stylet can be inserted through said lumen into said lead, said electrical contacts on said sheet on said connector being placed on either side of said lumen.

12. The implantable medical apparatus of claim 1 wherein said sheet has an upper side and a lower side, said electrical contacts being exposed through said lower side and protruding through said upper side, each of said electrical contacts being electrically coupled to a conductor of said lead near said upper side of said sheet, and wherein at least said upper side of said sheet is encapsulated in a biocompatible polymer.

13. The implantable medical apparatus of claim 1 wherein said electrical device comprises a cardiac stimulator having means for stimulating the heart of a patient, and wherein said lead has a plurality of electrodes near said distal end of said lead, each electrode electrically coupled to an electrical conductor.

14. A lead for an implantable medical apparatus, said lead comprising
an elongated lead body having a distal end and a proximal end,
a plurality of electrodes near said distal end of said lead body,
a plurality of electrical conductors, and
a connector coupled to a proximal end of said lead, said connector having a non-conducting sheet and a plurality of electrical contacts protruding from said sheet, each of said contacts being electrically connected to an electrical conductor in said lead.

15. The lead of claim 14 wherein each of said electrical contacts on said sheet comprise a pin extending through said sheet.

16. The lead of claim 15 wherein said pins comprise a bottom section and a flat section, said flat section extending above the sheet and being split by a slot, one of said conductors of said lead being inserted in said slot of a pin.

17. The lead of claim 16 wherein said flat section of said pins comprises a first tab and a second tab, said tabs being separated by said slot and said second tab being longer than said first tab.

18. The lead of claim 17 further comprising a cap on said first and second tabs, said cap clamping said conductor in said slot.

19. The lead of claim 15 wherein said pins comprise a cylindrical bottom section extending below the sheet, said cylindrical bottom section having a rounded distal end.

20. The lead of claim 14 further comprising a compressible seal circumferentially surrounding said electrical contacts on said sheet.

21. The lead of claim 14 wherein said connector further comprises a lumen opening into said lead body such that a stylet can be inserted through said lumen into said lead body and said electrical contacts on said sheet are disposed on either side of said lumen.

22. The lead of claim 14 wherein said sheet has an upper side and a lower side, said electrical contacts being exposed through said lower side and protruding through said upper side, each of said electrical contacts being electrically coupled to a conductor of said lead near said upper side of said sheet, and wherein at least said upper side of said sheet is encapsulated in a biocompatible polymer.
